(19) United States
(12) Patent Application Publication
(10) Pub. No.: US 2024/0404669 A1
(43) Pub. Date: Dec. 5, 2024

(54) PHYSICIAN ASSISTANT GENERATIVE MODEL

(71) Applicant: MDLIVE, Inc., Miramar, FL (US)

(72) Inventors: Michael Shope, Orlando, FL (US); Chuck B. Metturdharma, Bloomfield, CT (US); Lakshmikumari Meerasankaranarayanan, Bloomfield, CT (US); Saul Moncada, Miramar, FL (US)

(21) Appl. No.: 18/680,586

(22) Filed: May 31, 2024

Related U.S. Application Data
(60) Provisional application No. 63/470,726, filed on Jun. 2, 2023.

(51) Int. Cl. G16H 15/00; G16H 80/00

(52) U.S. Cl. CPC G16H 15/00; G16H 80/00

(57) ABSTRACT

Methods and systems for automatically generating physician documents are provided. The methods and systems receive a transcript representing a clinical encounter between a patient and a clinician. The methods and systems analyze the transcript using a generative machine learning model to automatically generate a preliminary post patient encounter document that comprises patient information and information about the clinical encounter. The methods and systems generate, for display to the clinician, a graphical user interface (GUI) comprising the automatically generated preliminary post patient encounter document.

### CROSS-REFERENCE TO RELATED APPLICATION(S)

This application claims the benefit of U.S. provisional patent application Ser. No. 63/470,726, filed 2 Jun. 2023, which is hereby incorporated by reference.

### BACKGROUND

Patient medical records are managed and generated in a variety of ways. Usually, the information contained in these records is manually entered and is prone to human error.

### BRIEF DESCRIPTION OF THE DRAWINGS

FIG. 1 is a block diagram of an example patient management platform, according to some examples.

FIG. 2 is an example database that may be deployed within the system of FIG. 1, according to some examples.

FIG. 3 is a block diagram of an example patient management platform that may be deployed within the system of FIG. 1, according to some examples.

FIG. 4 is a block diagram of an example implementation of a virtual visit patient management platform, according to some examples.

FIG. 5 is an example user interface of the patient management platform, according to some examples.

FIG. 6 is a flowchart illustrating example operations of the patient management platform, according to some examples.

FIG. 7 is a block diagram illustrating an example software architecture, which may be used in conjunction with various hardware architectures herein described.

FIG. 8 is a block diagram illustrating components of a machine, according to some examples.

FIG. 9 is a functional block diagram of an example neural network that can be used for the inference engine or other functions (e.g., engines) as described herein to produce a predictive model.

FIG. 10 is an example user interface of the patient management platform, according to some examples.

FIG. 11 is an example user interface of the patient management platform, according to some examples.

### DETAILED DESCRIPTION

Example methods and systems for a patient management platform are provided. Specifically, the methods and systems automatically generate post patient encounter documents representing a clinical encounter between a patient and a clinician based on a transcript of the clinical encounter. In the following description, for purposes of explanation, numerous specific details are set forth in order to provide a thorough understanding of the examples. It will be evident, however, to one of ordinary skill in the art that examples of the disclosure may be practiced without these specific details.

Virtual clinical encounters between clinicians and patients have become commonplace and are routinely performed. Following each of these encounters, the clinicians usually need to prepare documents, such as subjective, objective, assessment, plan (SOAP) notes among other documentation. These documents summarize the clinical encounter and include a variety of information about the patient and treatment plans. The process of generative these documents is incredibly complex and takes a great deal of meticulous effort and time. For example, the clinician usually has to navigate through multiple pages of information that includes notes about the clinical encounter and previous patient information which takes a great deal of time and effort. Also, because these documents are generated manually, they are prone to human error which can be propagated downstream to other medical services the patients seek and can introduce a variety of problems. For example, billing errors can result and, even worse, wrong prescriptions can be sent out.

The disclosed techniques provide systems and methods to automate the process of generating the post patient encounter documents, such as using a large language model (LLM) (e.g., a machine learning model, artificial neural network, a generative machine learning model, and so forth). The disclosed techniques receive a transcript representing a clinical encounter between a patient and a clinician. The disclosed techniques analyze the transcript using a generative machine learning model to automatically generate a preliminary post patient encounter document that includes patient information and information about the clinical encounter. The disclosed techniques generate, for display to the clinician, a graphical user interface (GUI) that includes the automatically generated preliminary post patient encounter document.

In some cases, the disclosed techniques present next to the automatically generated preliminary post patient encounter document a template for completing a final post patient encounter document. An example of a document used with the various embodiments described herein can be an electronic record that can be edited using a provider device or a clinician device with the electronic record being temporarily displayed at the provider/clinician device that can approve a final electronic record of the encounter to a system storage. The disclosed techniques can receive input from the clinician accepting/rejecting certain fields of the automatically generated preliminary post patient encounter document for populating (at least in part in a semi-automated manner) the final post patient encounter document. The proposed encounter document can include highlighted portions for the clinician to revise or to which the clinician should review in greater detail than other portions. The system can rank these highlighted portions based on a confidence score. The confidence score can be calculated using large language models, generative artificial intelligence or the like. As a result, a great deal of time and resources are saved and the user need not have to navigate through a multitude of pages of information to generate the final post patient encounter document. This saves time and reduces the amount of resources needed to accomplish a task. Also, because at least some of the information that is input in the final post patient encounter document is automatically generated, the risk of human errors being present in the final post patient encounter document is substantially reduced.

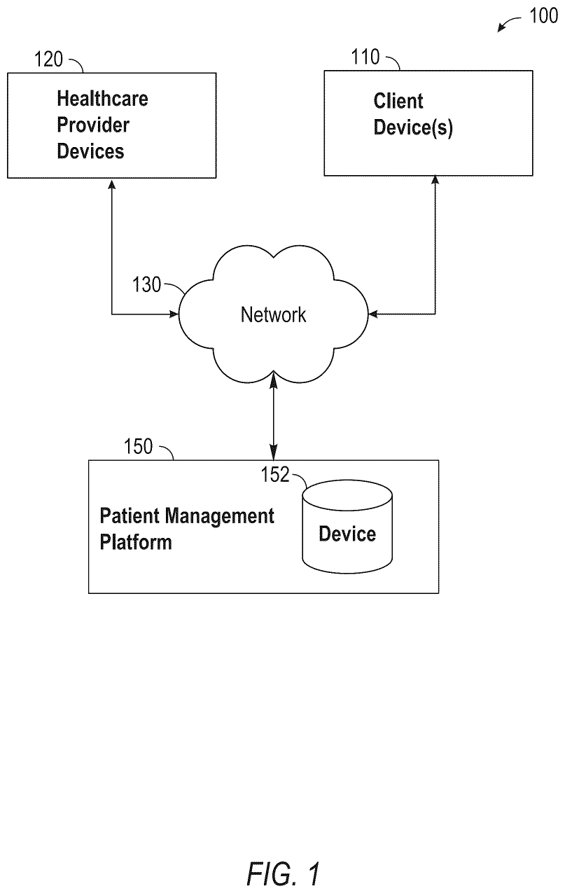

FIG. 1 is a block diagram showing an example patient management system 100, according to some examples. The patient management system 100 includes one or more client devices 110, one or more healthcare provider devices 120, and a patient management platform 150 that are communicatively coupled over a network 130 (e.g., Internet, telephony network).

As used herein, the term “client device” may refer to any machine that interfaces to a communications network (such as network 130) to access the patient management platform 150. The client device 110 may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network or the patient management platform 150. However, when the client device is loaded with instructions to perform the functions described herein, the client device is a dedicated machine.

In some cases, the patient management platform 150 is accessible over a global communication system, e.g., the Internet or world wide web. In such instances, the patient management platform 150 hosts a website that is accessible to the client devices 110. Upon accessing the website, the client devices 110 provide secure login credentials, which are used to access a profile associated with the login credentials and one or more patient profiles or patient information. As used herein, patient information includes any medical information associated with a patient including one or more prior medical insurance claims that were approved or denied, one or more electronic health records or medical health records, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, and/or one or more treatment preferences. One or more user interfaces associated with the patient management platform 150 are provided over the Internet via the website to the client devices 110.

Healthcare provider devices 120 can include the same or similar functionality as client devices 110 for accessing the patient management platform 150. In some cases, the healthcare provider devices 120 are used by “internal” users. Internal users are medical professionals, such as medical personnel, physicians, clinicians, healthcare providers, health-related coaches pharmacy benefit manager (PBM) operators, therapist, pharmacists, specialty pharmacy operators or pharmacists, or the like that are associated with, certified by, or employed by one or more organizations that provides the patient management platform 150. In some cases, the healthcare provider devices 120 are used by “external” users. External users are medical professionals and personnel, such as physicians, clinicians, and health-related coaches that are associated with or employed by a different (external) organization than that which provides the patient management platform 150.

The healthcare provider devices 120, when used by internal or external users, to access the patient management platform 150 can view many records associated with many different patients (or users associated with client devices 110) and their respective patient information. Different levels of authorization can be associated with different internal and different external users to control which records the internal and external users have access. In some instances, only records associated with those patients to which a given internal or external user is referred, are made accessible and available to the given internal or external user device. Sometimes, a first internal or external user can refer a patient or records associated with the patient to a second internal or external user. In such circumstances, the second internal or external user becomes automatically authorized to access and view the patient's records that were referred by the first internal or external user.

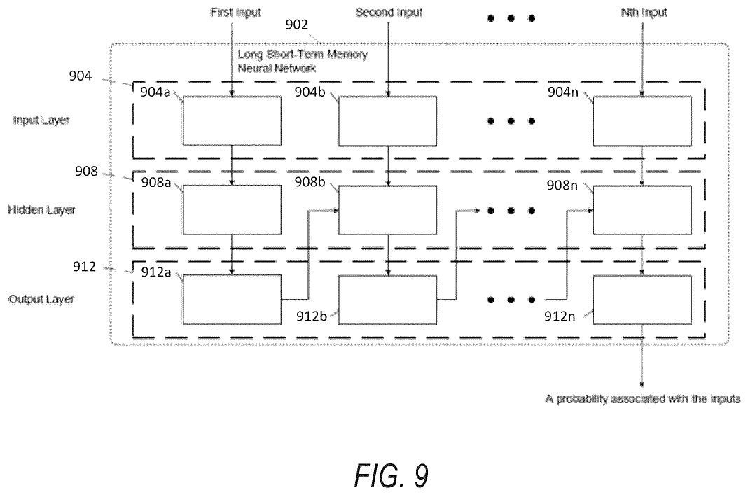

In some examples, the patient management platform 150 (and specifically the medical testing recommendation system 156) can implement a machine learning technique or machine learning model, such as a neural network (discussed below in connection with FIG. 9). The patient management platform 150 can use the machine learning model to simplify and expedite the management of patient information and the generation of post visit documents (e.g., SOAP notes). Particularly, the patient management platform 150 can be accessed by the healthcare provider devices 120. The patient management platform 150 can implement a first machine learning model that receives an audio file including contents of a clinical encounter between a clinician (or other medical professional or personnel) and a patient. The first machine learning model can generate a transcription of that clinical encounter. The output of the first machine learning model can be provided to a second machine learning model (e.g., an LLM, or other generative model), such as along with one or more prompts.

In some examples, the second machine learning model can automatically generate a preliminary post patient encounter document (e.g., a preliminary SOAP note). While the disclosed examples are provided in connection with SOAP notes, similar techniques can be applied to any other type of post patient encounter document, such as a problem, intervention, and evaluation (PIE) note, a data, assessment, and plan (DAP) note, a behavior, intervention, response, and plan (BIRP) note, a follow-up, outcomes, care, upcoming visits, and symptoms (FOCUS) note, or a chief compliant, history, assessment, treatment, and test results (CHART) note, or an intervention, assessment, and/or plan (IAP) note. The preliminary post patient encounter document can then be presented to the clinician on the healthcare provider devices 120 in a GUI. The clinician can use the preliminary post patient encounter document presented in the GUI to populate a template for a final post patient encounter document (e.g., a final SOAP note). In some cases, tracking information is maintained that determines which portions of the preliminary post patient encounter document have been accepted and which have been rejected. This tracking information can be used to control further training of the second machine learning model to improve generation of subsequent preliminary post patient encounter documents.

Specifically, the second machine learning model can be trained to establish a relationship between patterns of a plurality of clinical encounter transcripts and patterns of post patient encounter documents. The second machine learning model can then receive a new transcript of a clinical encounter and can estimate, generate or predict a preliminary post patient encounter document for that clinical encounter represented by the new transcript. This allows the clinician accessing the preliminary post patient encounter document a more efficient and effective and accurate way to generate a final post patient encounter document. This reduces the number of times information needs to be repetitively copied, improves the overall accuracy of generating post patient encounter document, and reduces the number of pages of information and interfaces the clinician has to navigate through to generate the post patient encounter document.

In some examples, the second machine learning model can be trained by obtaining a batch of training data comprising a first set of the patterns of the plurality of clinical encounter transcripts. The second machine learning model processes the first set of the patterns of the plurality of clinical encounter transcripts by the LLM to generate an estimated set of post patient encounter documents. The second machine learning model computes a loss based on a deviation between the estimated set of post patient encounter documents and the patterns of post patient encounter documents associated with the first set of the patterns of the plurality of clinical encounter transcripts. The second machine learning model updates one or more parameters of the LLM based on the computed loss.

The second machine learning model (e.g., the artificial network including an LLM, or other diffusion network) can be used for SOAP note generation from transcriptions of clinical encounters by training the second machine learning model on a large dataset of clinical encounter transcriptions and their corresponding ground truth SOAP notes (the actual SOAP notes written by the clinicians for each clinical encounter associated with the transcription). The second machine learning model learns the statistical relationships between the transcriptions and their corresponding ground truth SOAP notes. During training, the second machine learning model learns to generate a sequence of SOAP notes by iteratively refining them with multiple rounds of stochastic diffusion steps. The second machine learning model starts with a random noise vector and applies a series of diffusion steps to iteratively refine the SOAP note. At each diffusion step, the second machine learning model applies a random noise to the SOAP note and then calculates the gradients of the SOAP note with respect to the loss function. The gradients are then used to update the SOAP note, which is then further refined in the next diffusion step.

The second machine learning model generates SOAP notes by sampling from the sequence of SOAP note samples produced during the diffusion process. The second machine learning model uses a learned autoregressive model to generate each header and portion of the SOAP note, conditioned on the ground-truth SOAP note. Overall, the process of generating SOAP notes from text or transcriptions using the second machine learning model involves training the second machine learning model on a large dataset of SOAP notes and transcriptions, and then using the trained second machine learning model to generate SOAP notes by sampling from the learned distribution of images.

Specifically, the second machine learning model preprocesses the SOAP notes of a training set that includes transcriptions and ground truth SOAP notes. This may involve normalizing the SOAP notes and splitting the data into training and validation sets. The second machine learning model is trained on the training data using an encoder that encodes the transcriptions into a low-dimensional vector space, a generator that generates SOAP notes from noise vectors, and a discriminator that distinguishes between real and generated SOAP notes. The second machine learning model, during training, is trained to minimize a loss function that encourages the generated SOAP notes to match the real SOAP notes (e.g., the ground truth SOAP notes of the same transcription). The loss function can consist of a combination of adversarial loss, reconstruction loss, and textual consistency loss.

Once the second machine learning model is trained, SOAP notes can be generated from transcriptions of clinical encounters by sampling from the learned distribution of SOAP notes conditioned on the transcriptions and/or prompts. The training operations can operate on additional sets of training images until the stopping criterion is satisfied or reached. This results in the generation of high-quality SOAP notes that are closely aligned with the corresponding ground-truth SOAP notes.

In some examples, the patient management platform 150 presents in the GUI a plurality of fields of the automatically generated preliminary post patient encounter document. The patient management platform 150 receives input from the clinician selecting data from the plurality of fields. The patient management platform 150 populates a final post patient encounter document in response to receiving the input.

In some examples, the patient management platform 150 receives a selection of an accept option associated with a first field of the plurality of fields. The patient management platform 150 automatically transfers data from the first field to a corresponding field of the final post patient encounter document in response to receiving the selection. In some cases, the patient management platform 150 receives a selection of a rejection option associated with a first field of the plurality of fields. The patient management platform 150 identifies a corresponding field of the final post patient encounter document corresponding to the first field in response to receiving the selection. The patient management platform 150 generates training data including a difference between data in the corresponding field and data in the first field.

In some examples, the patient management platform 150 generates a tracking report indicating which fields of the plurality of fields have been accepted and which fields of the plurality of fields have been rejected. In some cases, the patient management platform 150 updates the generative machine learning model based on the tracking report.

In some examples, the tracking report represents an acceptance rate of the plurality of fields across multiple automatically generated preliminary post patient encounter documents representing multiple clinical encounters. The patient management platform 150 measures an acceptance rate associated with respective fields of the multiple automatically generated preliminary post patient encounter documents. The patient management platform 150 determines that the acceptance rate fails to transgress a threshold and triggers updating the generative machine learning model based on the tracking report in response to determining that the acceptance rate fails to transgress the threshold.

In some examples, the automatically generated preliminary post patient encounter document includes a JSON file. In some examples, the patient management platform 150 establishes a virtual visit for conducting the clinical encounter between the clinician and the patient. The patient management platform 150 generates an audio recording of the clinical encounter conducted in the established virtual visit and processes the audio recording by a machine learning model to generate the transcript.

In some examples, the patient management platform 150 generates one or more prompts for generating the preliminary post patient encounter document. The patient management platform 150 provides the one or more prompts and the transcript to the generative machine learning model to automatically generate the preliminary post patient encounter document. In some cases, the patient management platform 150 receives permission from the patient approving the generating of the audio recording and the analyzing of the transcript by the generative machine learning model.

In some examples, the virtual visit is established by a first server, the transcript is encrypted before being provided to the generative machine learning model, and the generative machine learning model is accessed by a second server after conducting authentication between the first server and the second server.

The network 130 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The healthcare provider devices 120 can be used to access pharmacy claims, medical data (e.g., medical information 230 stored in database 152), laboratory data and the like for one or more patients that the healthcare provider devices 120 are authorized to view. This patient information 210 can be maintained in a database 152 by the patient management platform 150 or in a third-party database accessible to the patient management platform 150 and/or the healthcare provider devices 120.

In some examples, the client devices 110 and the patient management platform 150 can be communicatively coupled via an audio call (e.g., VoIP, Public Switched Telephone Network, cellular communication network, etc.), video call, or via electronic messages (e.g., online chat, instant messaging, text messaging, email, and the like). In some examples, the client devices 110 communicate directly or indirectly with the healthcare provider devices 120 to establish and conduct a virtual clinical encounter (video-based and/or audio-based clinical encounter) between a patient and a clinician (or other medical professional). The client devices 110 can present a GUI to the patient asking the patient to consent to the virtual clinical encounter being recorded, transcribed, and/or processed by the generative machine learning model for generating/producing a post visit document (e.g., a SOAP note).

While FIG. 1 illustrates a single client device 110 and a single healthcare provider device 120, it is understood that a plurality of such devices can be included in the system 100 in other examples.

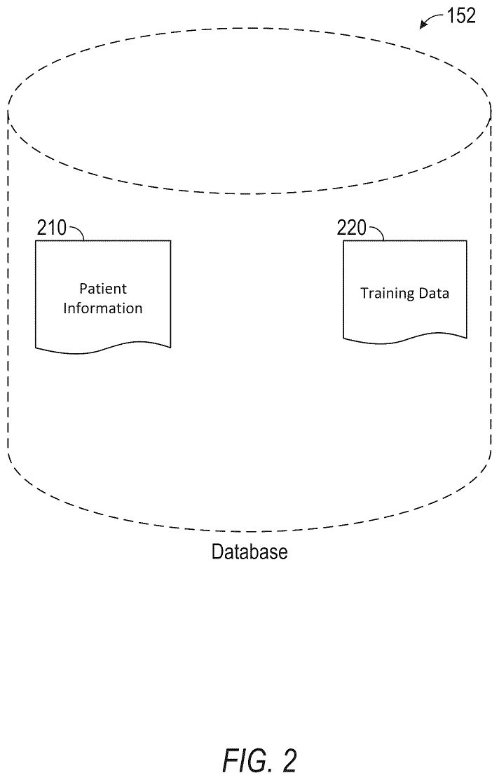

FIG. 2 is an example database 152 that may be deployed within the system of FIG. 1, according to some examples. The database 152 includes patient information 210 and training data 220. The patient information 210 can be generated or accessed by the patient management platform 150. The patient information 210 can include a transcription of one or more virtual clinical encounters. For example, the patient management platform 150 can access one or more patient records from one or more sources, including pharmacy claims, benefit information, prescribing physician information, dispensing information (e.g., where and how the patient obtains their current medications), demographic information, prescription information including dose quantity and interval, and input from a patient received via a user interface presented on the client device 110 and so forth. The patient management platform 150 can collect this information from the patient records and generates a patient features vector that includes this information.

The training data 220 includes training sets including multiple sets of a plurality of training transcriptions or features of such transcriptions with respective sets of ground-truth post patient encounter documents (e.g., SOAP notes). The training data 220 is used to train a machine learning model (e.g., an LLM model) implemented by patient management platform 150 to generate preliminary post patient encounter documents. For example, the training data 220 can be built over time by analyzing user behavior and interactions with various preliminary patient encounter documents (e.g., indicating which portions of the preliminary patient encounter documents were accepted to be incorporated into the final patient encounter documents and which were rejected) and/or final patient encounter documents associated with training transcriptions of virtual clinical encounters.

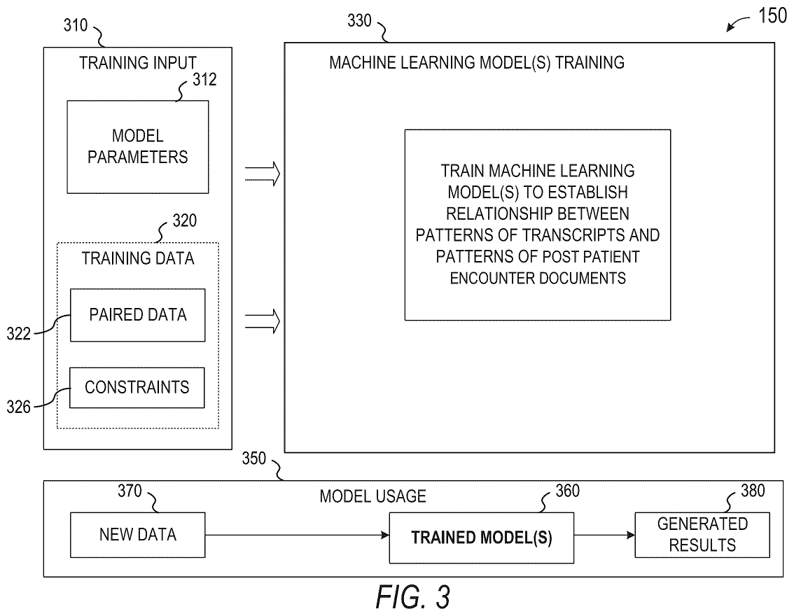

FIG. 3 is a block diagram of an example service of patient management platform 150 that may be deployed within the system of FIG. 1, according to some examples. Training input 310 includes model parameters 312 and training data 320 (e.g., training data 220 (FIG. 2)) which may include paired training data sets 322 (e.g., input-output training pairs) and constraints 326. Model parameters 312 stores or provides the parameters or coefficients of corresponding ones of machine learning models. During training, these parameters 312 are adapted based on the input-output training pairs of the training data sets 322. After the parameters 312 are adapted (after training), the parameters are used by trained models 360 to implement the trained machine learning models on a new set of data 370.

Training data 320 includes constraints 326 which may define the constraints of a given patient information features. The paired training data sets 322 may include sets of input-output pairs, such as pairs of a plurality of training virtual clinical encounter transcription features and features of post patient encounter documents that are created in association with one or more of the training transcriptions (e.g., ground-truth patient encounter documents). Some components of training input 310 may be stored separately at a different off-site facility or facilities than other components.

Machine learning model(s) training 330 trains one or more machine learning techniques based on the sets of input-output pairs of paired training data sets 322. For example, the model training 330 may train the machine learning (ML) model parameters 312 by minimizing a loss function based on one or more ground-truth patient encounter documents generated in association with a training transcription. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, an LLM, a generative network, a diffusion model, and the like.

Particularly, the ML model can be applied to a training batch of transcription features to estimate or generate one or more preliminary patient encounter documents. In some implementations, a derivative of a loss function is computed based on a comparison of the preliminary patient encounter documents and the ground truth patient encounter documents associated with the training transcription features and parameters of the ML model are updated based on the computed derivative of the loss function.

The result of minimizing the loss function for multiple sets of training data trains, adapts, or optimizes the model parameters 312 of the corresponding ML models. In this way, the ML model is trained to establish a relationship between a plurality of training transcriptions and ground-truth patient encounter documents associated with the training transcriptions.

After the machine learning model is trained, new data 370, including one or more transcriptions of a virtual clinical encounter features are received and/or derived by the patient management platform 150. The trained machine learning model may be applied to the new data 370 to generate results 380 including a prediction of preliminary patient encounter documents. The preliminary patient encounter documents can be represented in a GUI, such as in a prompt overlaid on the GUI allowing a clinician to selectively include or exclude portions of the preliminary patient encounter documents in a final patient encounter document.

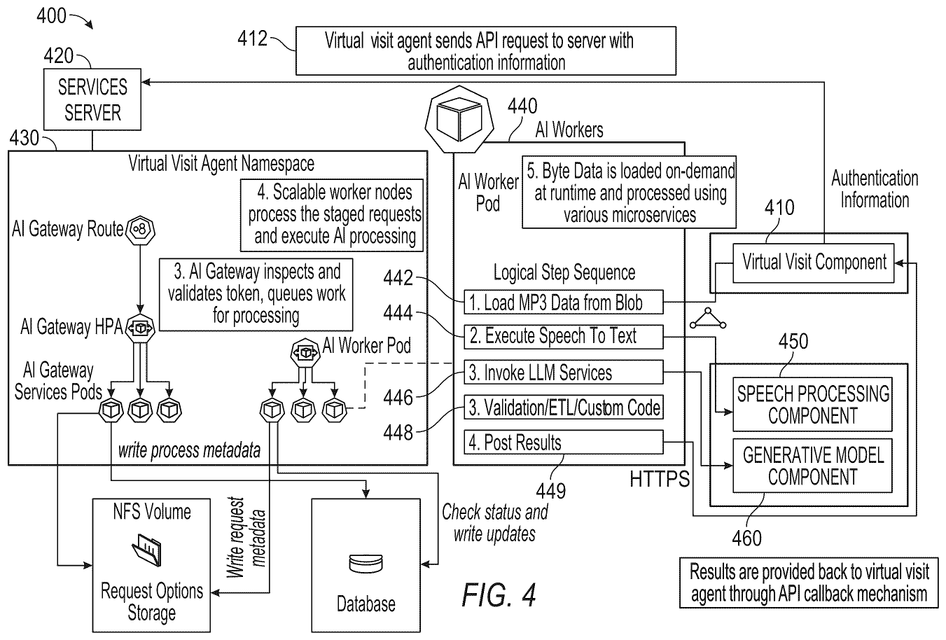

FIG. 4 is a block diagram of an example implementation of a virtual visit patient management platform 400, according to some examples. Some or all of the components shown in FIG. 4 can be included as part of the patient management platform 150 and vice versa. The virtual visit patient management platform 400 can include a services server 420, a virtual visit component 410, one or more artificial intelligence (AI) workers component 440, a virtual visit agent namespace 430, a speech processing component 450, and/or a generative model component 460.

In some examples, the virtual visit component 410 can enable a patient to establish a communication session (virtual video and/or audio session) with a clinician (e.g., medical professional). The communication session can couple a device of the patient with a device of the clinician to enable the clinician to perform and conduct a clinical encounter with the patient. The virtual visit component 410 can present a GUI to the patient informing the patient about the identity of the clinician. The virtual visit component 410 can present in the GUI a notification requesting approval from the patient to have the clinical encounter recorded. In response to receiving an approval from the patient (or automatically without receiving input from the patient), the virtual visit component 410 can begin recording video and/or audio of the clinical encounter.

The virtual visit component 410 can detect that the clinical encounter has been terminated, such as when the communication session is closed between the clinician and the patient. In such cases, the virtual visit component 410 can stop recording the clinical encounter and can generate and store a file that includes the video and/or audio of the clinical encounter. The file can be encrypted by the virtual visit component 410. The virtual visit component 410 can establish a secure connection by sending authentication information to the services server 420. In some cases, the virtual visit component 410 sends an API request 412 to the services server 420 to perform further processing of the encrypted file.

The virtual visit component 410, after establishing the secure communication session with the services server 420, provides the encrypted file to the services server 420. The services server 420 identifies a virtual visit agent namespace 430 associated with the virtual visit component 410. This virtual visit agent namespace 430 can include various services that are all securely contained under various encryption protocols and are accessible exclusively by the virtual visit component 410. The virtual visit agent namespace 430 can be implemented by servers that are remote from the virtual visit component 410 and provide services and functions to the virtual visit component 410 in a secure manner.

The services in the virtual visit agent namespace 430 can decrypt the file received from the virtual visit component 410. The virtual visit agent namespace 430 can then call on the AI workers component 440 to perform various operations and analysis of the unencrypted file. For example, the virtual visit agent namespace 430 can load the file in step 442 and then execute a speech to text agent 444 on the unencrypted file. The speech to text agent 444 can provide the unencrypted file to the speech processing component 450. The speech processing component 450 processes the unencrypted file and generates a transcription of the contents in the file. The transcription can include metadata that defines various behavior attributes present in video frames when the file contains video of the clinical encounter. The speech processing component 450 can implement one or more machine learning models that are trained to generate transcriptions (e.g., medical transcription) from audio and/or video files received by the speech processing component 450.

The workers component 440 receives the transcription from the speech processing component 450. The workers component 440 can then retrieve one or more prompts associated with generation of a post visit document (e.g., a SOAP note). The workers component 440 provides the one or more prompts along with the transcription in step 446 to one or more LLM services. The LLM services can include the generative model component 460. The generative model component 460 receives the one or more prompts along with the transcription and automatically generates a preliminary post visit document that includes at least some of the patient information and summary information about the clinical encounter. The preliminary post visit document is provided to the AI workers component 440 and are validated at step 448 (e.g., using additional machine learning models). The validated preliminary post visit document is then provided as results 449 to the virtual visit component 410.

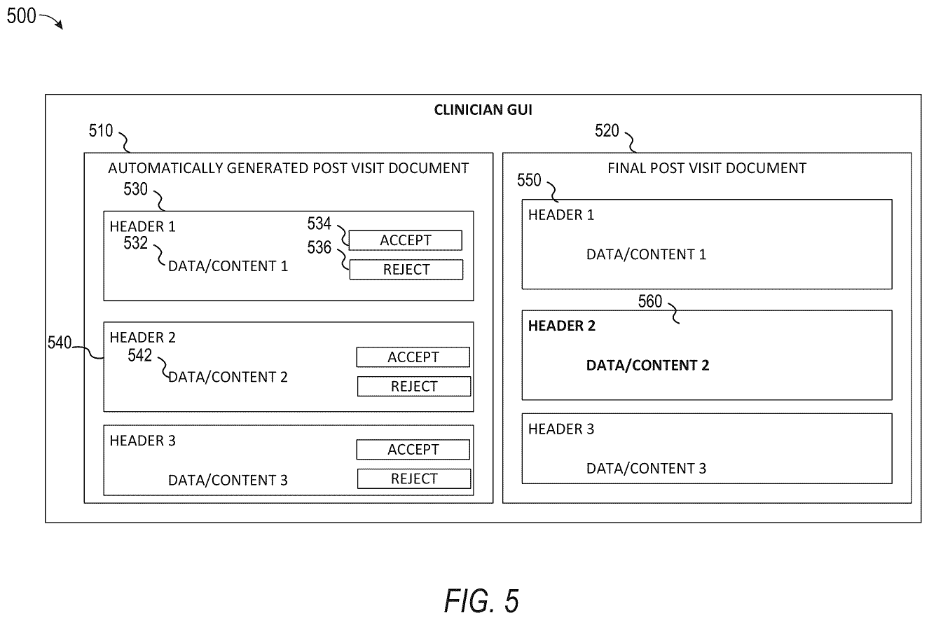

The virtual visit component 410 can present a GUI to the clinician that includes the results 449. In some cases, the results 449 are presented as a JSON file or HTML document with various headers or fields representing different components of the final post visit document. FIG. 5 is an example user interface 500 of the patient management platform 150 (and/or the virtual visit patient management platform 400), according to some examples. The user interface 500 can be presented on the healthcare provider devices 120 of the clinician who conducted the clinical encounter with the patient.

The graphical user interface 500 can be unique to each patient visit or unique to each type of patient visit, e.g., depending on the type of visit or the medical condition discussed during the telehealth visit. The present system can determine the visit type based on the answers from a patient client device during triage. In some examples, the present system identifies the visit type and assigns certain data types that should be included in the post visit document. The present system can identify each enter needed for a visit type. The fields can be automatically populated with data derived form at least one of the initial data for the patient from before the visit, processing audio from the telehealth visit (e.g., in real time during the visit or with a about minute+/−10 seconds, 30 seconds, 60 seconds, 90 seconds from the conclusion of the telehealth visit). In an example, if the present system will not provide the medical provider the suggested data for the final post visit document data record, the clinician GUI 500 will provide an estimate of the time it will take to provide the first portion fields with the headers and data/content. The time estimate can be based on the length of the telehealth visit, the completeness of the triage data from the patient prior to the telehealth visit, the predicted type of visit, language (English, Spanish, French, other languages, or combinations thereof). The provider needs not wait until the automatically generated post visit content is displayed to begin entry of their post visit notes. The clinician GUI 500 can have a third field that allows the provider to begin entering their own post visit data. This post visit data can be transmitted from the provider client device to the server to be used as input to the intelligence engine to be incorporated into the first portion 510 of the automatically generated preliminary post visit document 500.

The user interface 500 can present in a first portion 510 of the automatically generated preliminary post visit document provided as the results 449. The user interface 500 can present in a second portion 520 a template for populating a final post visit document. The first portion 510 can include a first field 530 corresponding to a first header of the final post visit document (e.g., the final SOAP note). The first field 530 can identify the first header by title and can include artificially and automatically generated data/content items 532. The virtual visit component 410 can receive, via the healthcare provider devices 120, a selection of an accept option 534 or a selection of a rejection option 536.

In some examples, in response to receiving the selection of the accept option 534, the virtual visit component 410 can copy the data/content items 532 to the corresponding field 550 of the second portion. The healthcare provider devices 120 can receive additional input modifying or editing the corresponding field 550 after the data/content items 532 are copied over to the corresponding field 550. In some examples, in response to receiving the selection of the reject option 536, the virtual visit component 410 can present a prompt that allows the clinician to input information to populate the corresponding field 550.

The virtual visit component 410 can track which information is copied over and accepted by the clinician and which information is rejected by the clinician in a tracking file. The tracking file can be kept for a large set of different automatically generated post visit documents that are presented to various clinicians. The virtual visit component 410 can track the acceptance rate based on the quantity of automatically generated post visit documents that are presented to clinicians and the relative number of times the accept options were selected. If the acceptance rate falls below a threshold, the virtual visit component 410 can call an API of the services server 420 to retrain and/or update one or more prompts used by the generative model component 460 to generate the post visit documents. In some cases, the virtual visit component 410 generates additional training data based on actual updated information provided by and received from the clinician in the process of completing the final post visit document. This additional training data can be used to update parameters and retrain the generative model component 460.

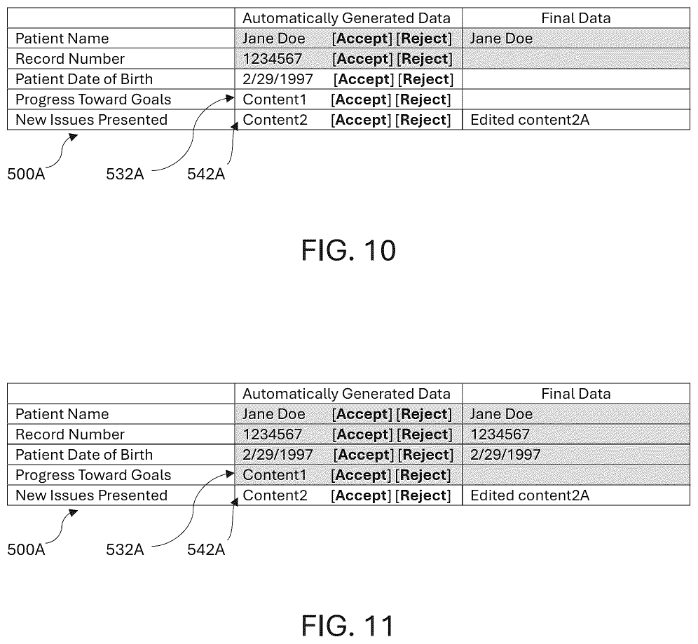

In some examples, a second field 540 associated with a second header 560 of the final post visit document can be presented. The healthcare provider devices 120 can receive input that accepts or rejects the data/content item 542 presented in the second field 540. The virtual visit component 410 can populate the second header 560 based in part on the acceptance or rejection of the data/content item 542. In some examples, the virtual visit component 410 can visually distinguish fields or portions of the final post visit document that were accepted by the clinician from those fields that were rejected by the clinician. For example, fields or headers of the final post visit document shown in bold can represent data/content items that were copied over from the automatically generated post visit document fields. Fields or headers of the final post visit document shown in regular font or a different color (e.g., different data box fill colors) can represent data/content items that were not copied over from the automatically generated post visit document fields and were actually manually or partially manually entered by the clinician using the client device. In an example, the fields that are copied from field 530 (or 540) to the field 550 (or 560), then the fields are filled with a same color. If the data in the field 550 is copied and then edited, then the field 550 can be filled with a color different than that color used when the data from the automatically generated post visit content fields is exactly copied to the final post visit fields. With reference to FIGS. 10-11, a provider/clinician GUI 500A is shown with the patient name field and the record number field in the automatically generated highlighted in grey showing that this data has been accepted using the client device of the provider/clinician and copied from the automatically generated fields to the final data fields. With reference to FIG. 11, the data in the date of birth fields and the progress toward goals field 532A content1 are also accepted using the client device of the provider/clinician and copied from the automatically generated fields to the final data fields. However, the provider/clinician did not accept the content2 in field 542A. Hence associated the final data field for the New Issues Presented is not highlighted in grey like the other fields. While shown in grey for ease of illustration, other indicating graphical features can be used to indicate which fields have been edited by provider through their client device. The GUI 500A will change based on the type of visit, which in this case can be a behavioral visit, a physical therapy visit, or a follow up visit for a medical condition. In the case of this being an initial telehealth visit for a patient, the progress toward goals fields in the middle and right columns are removed.

The automatically generated post visit fields can also include an image of a human body or an image of the body part that is subject to the visit. The image can have the body part discussed during the visit indicated automatically, e.g., using the LLM engine or generative artificial intelligence engine described herein. Example implementations can have the body part discussed shown in solid line, while the non-discussed body parts shown in broken line, an arrow pointing to the discussed body part, the body part discussed circled or surrounded by another geometric shape, or the like.

The clinician graphical user interface 500 includes input fields in the first portion 510 and the second portion 520. The text in the fields of the second portion of the clinician GUI are editable by the provider. The text in the fields of the first portion 510 are not editable in these fields but can be edited once the content is accepted and copied to the fields in the second portion 520. The headers for each field can be produced by the system and methods described herein with the headers being selected based on the visit type, e.g., ear infection, sore throat, eye infection, muscular skeletal pain, behavioral health, psychological, dermatology, wellness visit, UTI visit, allergies, etc. In a simple form, the first heading can be a patient information, e.g., name and date of birth, the first field 530 can have a heading of Subjective with data/content 1 532 being populated with data derived from telehealth visit, the second field 540 can have a heading Objective with the data/content2 542 being populated with data derived from telehealth visit, a third field can be Assessment with its data/content3 being populated with data derived from telehealth visit, a fourth field (not shown in FIG. 5) can have a heading of Plan with its data/content being derived from the telehealth visit. The layout of the clinician graphical user interface 500 changes based on the type of visit and the number of fields needed to complete the post visit record. The layout of the clinician graphical user interface 500 can also change based on interaction of the provider with client device, e.g., once the data/content is accepted and copied to fields in the post visit portion 520, these fields can collapse to a single line allowing for other fields to be pulled upward on the display and reducing the need to scroll down to see all of the fields or all of the content/data in the fields.

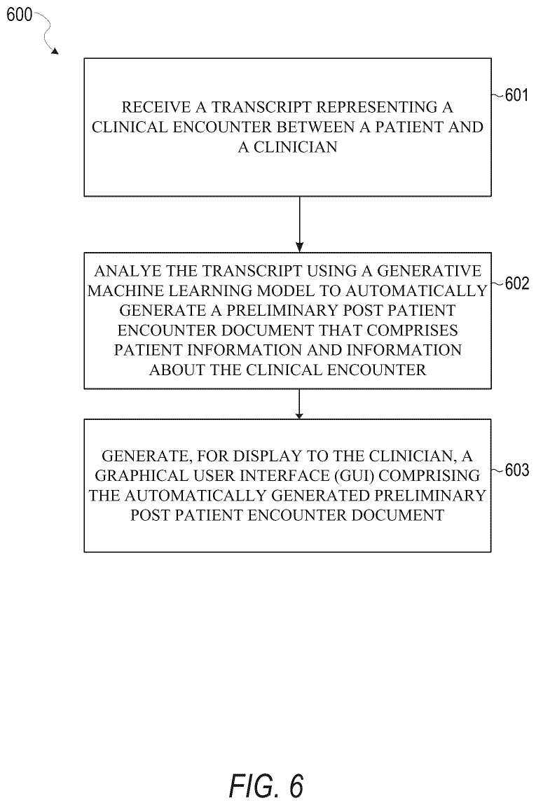

FIG. 6 is a flowchart illustrating example operations and methods of the patient management platform 150 in performing a method or process 600, according to some examples. The process 600 may be embodied in computer-readable instructions for execution by one or more processors such that the operations of the process 600 may be performed in part or in whole by the functional components of the system 100; accordingly, the process 600 is described below by way of example with reference thereto. However, in other examples, at least some of the operations of the process 600 may be deployed on various other hardware configurations. Some or all of the operations of process 600 can be in parallel, out of order, or entirely omitted.

At operation 601, the system 100 receives a transcript representing a clinical encounter between a patient and a clinician, as discussed above.

At operation 602, the system 100 analyzes the transcript using a generative machine learning model to automatically generate a preliminary post patient encounter document that comprises patient information and information about the clinical encounter, as discussed above.

At operation 603, the system 100 generates, for display to the clinician, a graphical user interface (GUI) comprising the automatically generated preliminary post patient encounter document, as discussed above.

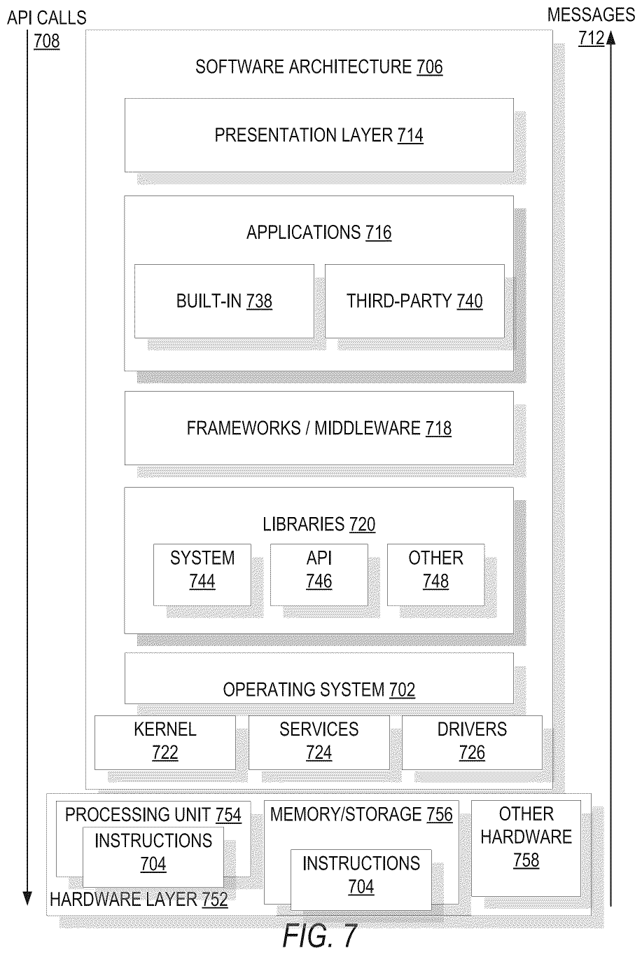

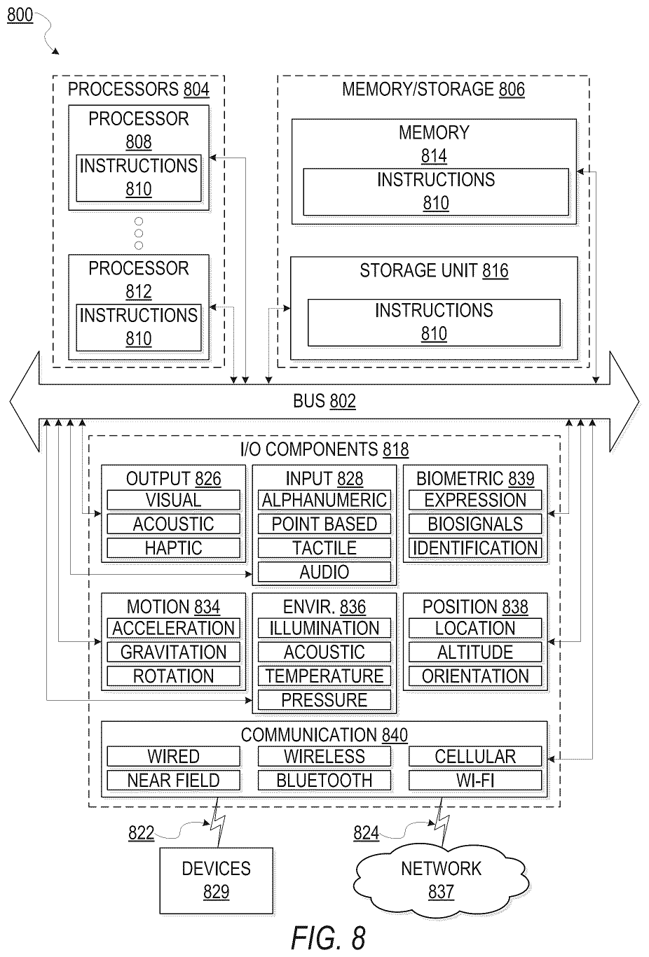

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 1. The software architecture 706 can be utilized to apply a machine learning technique or model to generate a prediction of preliminary post patient encounter documents based on a transcription of a clinical encounter.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some examples, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the system 100 to process fields of a transcript being accessed by the patient management platform 150 with a trained machine learning model to predict one or more preliminary post patient encounter documents.

As such, the instructions 810 may be used to implement devices or components described herein. The instructions 810 transform the general, non-programmed machine 800 into a particular machine 800 programmed to carry out the described and illustrated functions in the manner described. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, database 152, and a storage unit 816, both accessible to the processors 804 such as via the bus 802. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements, and so on. The specific I/O components 818 that are included in a particular machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further examples, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting a NFC beacon signal that may indicate a particular location, and so forth.

The machines 700 and 800 described in FIGS. 7 and 8 are be dedicated machines for providing telehealth and determining content for providers as discussed herein. The machines 700, 800 can automatically find relationships between data from a telehealth visit to suggest content for documenting a provider record or a patient record. The machines 700, 800 can run intelligent tools including generative artificial intelligence, large language models, machine learning that uses algorithms to generate new content, code, or data relating to telehealth visits, within the context of the present disclosure. Generative AI tools can reside and run on the machines 700, 800. The tasks for the machines can be in one or more computer programs configured to perform particular operations or actions by virtue of including instructions that, when executed by machines 700, 800, cause the machine to perform the actions. Such programs run on dedicated circuitry.

FIG. 9 is a functional block diagram of an example neural network 902 that can be used for the inference engine or other functions (e.g., engines) as described herein to produce a predictive model. The predictive model can identify or generate a prediction of one or more preliminary post patient encounter document in association with one or more transcripts of a clinical encounter. In an example, the neural network 902 can be a LSTM neural network. In an example, the neural network 902 can be a recurrent neural network (RNN). The example neural network 902 may be used to implement the machine learning as described herein, and various implementations may use other types of machine learning networks. The neural network 902 includes an input layer 904, a hidden layer 908, and an output layer 912. The input layer 904 includes inputs 904a, 904b . . . 904n. The hidden layer 908 includes neurons 908a, 908b . . . 908n. The output layer 912 includes outputs 912a, 912b . . . 912n.

Each neuron of the hidden layer 908 receives an input from the input layer 904 and outputs a value to the corresponding output in the output layer 912. For example, the neuron 908a receives an input from the input 904a and outputs a value to the output 912a. Each neuron, other than the neuron 908a, also receives an output of a previous neuron as an input. For example, the neuron 908b receives inputs from the input 904b and the output 912a. In this way the output of each neuron is fed forward to the next neuron in the hidden layer 908. The last output 912n in the output layer 912 outputs a probability associated with the inputs 904a-904n. Although the input layer 904, the hidden layer 908, and the output layer 912 are depicted as each including three elements, each layer may contain any number of elements. Neurons can include one or more adjustable parameters, weights, rules, criteria, or the like.

In various implementations, each layer of the neural network 902 must include the same number of elements as each of the other layers of the neural network 902. For example, training GUI features (e.g., fields of a GUI presented to an operator) may be processed to create the inputs 904a-904n. The neural network 902 may implement a model to produce one or more preliminary post patient encounter document in association with the transcript features. More specifically, the inputs 904a-904n can include fields of the transcript as data features (binary, vectors, factors or the like) stored in the storage device 110. The fields of the transcript as data features can be provided to neurons 908a-908n for analysis and connections between the known facts. The neurons 908a-908n, upon finding connections, provides the potential connections as outputs to the output layer 912, which determines a preliminary post patient encounter document.

The neural network 902 can perform any of the above calculations. The output of the neural network 902 can be used to trigger display of a prompt that includes the preliminary post patient encounter document in a GUI. For example, the prompt (e.g., notification) can be provided to a PBM, health plan manager, pharmacy, physician, clinician, caregiver, and/or a patient.

In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 904a is connected to each of neurons 908a, 908b . . . 908n.

### Glossary

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols. The carrier signal can connect the client device to the system described herein over the communication network in a secure manner, e.g., to have a telehealth visit.

“CLIENT DEVICE” in this context refers to any machine that interfaces to a communications network to obtain resources from one or more server systems or other client devices. A client device may be, but is not limited to, a mobile phone, desktop computer, laptop, PDA, smart phone, tablet, ultra-book, netbook, laptop, multi-processor system, microprocessor-based or programmable consumer electronics, game console, set-top box, or any other communication device that a user may use to access a network.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

Changes and modifications may be made to the disclosed techniques without departing from the scope of the present disclosure. These and other changes or modifications are intended to be included within the scope of the present disclosure, as expressed in the following claims.

The present application is generally related to the pending U.S. Provisional Application No. 63/471,194, filed 5 Jun. 2023, titled ARTIFICIAL-INTELLIGENCE-ASSISTED CONTENT PROCESSING OF CROSS-NETWORK COMMUNICATIONS, which is hereby incorporated by reference.

The Abstract of the Disclosure is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A method comprising:
receiving a transcript representing a clinical encounter between a patient and a clinician;
analyzing the transcript using a generative machine learning model to automatically generate a preliminary post patient encounter document that comprises patient information and information about the clinical encounter; and
generating, for display to the clinician, a graphical user interface (GUI) comprising the automatically generated preliminary post patient encounter document.

2. The method of claim 1, wherein the preliminary post patient encounter document comprises a subjective objective assessment plan (SOAP) note, a problem, intervention, and evaluation (PIE) note, a data, assessment, and plan (DAP) note, a behavior, intervention, response, and plan (BIRP) note, a follow-up, outcomes, care, upcoming visits, and symptoms (FOCUS) note, or a chief compliant, history, assessment, treatment, and test results (CHART) note, or an intervention, assessment, and plan (IAP) note.

3. The method of claim 1, wherein the clinical encounter comprises a virtual office visit, the clinician comprising a physician or therapist.

4. The method of claim 1, wherein the patient information comprises an electronic health record, past claims information for the patient, patient health information, past medical recommendations, past treatment recommendations, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, or one or more treatment preferences.

5. The method of claim 1, further comprising:
presenting in the GUI a plurality of fields of the automatically generated preliminary post patient encounter document;
receiving input from the clinician selecting data from the plurality of fields; and
populating a final post patient encounter document in response to receiving the input.

6. The method of claim 5, further comprising:
receiving a selection of an accept option associated with a first field of the plurality of fields; and
automatically transferring data from the first field to a corresponding field of the final post patient encounter document in response to receiving the selection.

7. The method of claim 6, further comprising:
receiving a selection of a rejection option associated with a first field of the plurality of fields;
identifying a corresponding field of the final post patient encounter document corresponding to the first field in response to receiving the selection; and
generating training data comprising a difference between data in the corresponding field and data in the first field.

8. The method of claim 7, further comprising:
generating a tracking report indicating which fields of the plurality of fields have been accepted and which fields of the plurality of fields have been rejected.

9. The method of claim 8, further comprising updating the generative machine learning model based on the tracking report.

10. The method of claim 9, wherein the tracking report represents an acceptance rate of the plurality of fields across multiple automatically generated preliminary post patient encounter documents representing multiple clinical encounters, further comprising:
measuring an acceptance rate associated with respective fields of the multiple automatically generated preliminary post patient encounter documents;
determining that the acceptance rate fails to transgress a threshold; and
triggering updating the generative machine learning model based on the tracking report in response to determining that the acceptance rate fails to transgress the threshold.

11. The method of claim 1, wherein the automatically generated preliminary post patient encounter document comprises a JSON file.

12. The method of claim 1, further comprising:
establishing a virtual visit for conducting the clinical encounter between the clinician and the patient;
generating an audio recording of the clinical encounter conducted in the established virtual visit; and
processing the audio recording by a machine learning model to generate the transcript.

13. The method of claim 12, further comprising:
generating one or more prompts for generating the preliminary post patient encounter document; and
providing the one or more prompts and the transcript to the generative machine learning model to automatically generate the preliminary post patient encounter document.

14. The method of claim 13, further comprising:
receiving permission from the patient approving the generating of the audio recording and the analyzing of the transcript by the generative machine learning model.

15. The method of claim 13, wherein the virtual visit is established by a first server, wherein the transcript is encrypted before being provided to the generative machine learning model, and wherein the generative machine learning model is accessed by a second server after conducting authentication between the first server and the second server.

16. The method of claim 1, wherein the generative machine learning model comprises a large language model (LLM), and wherein the generative machine learning model is trained to establish a relationship between patterns of a plurality of clinical encounter transcripts and patterns of post patient encounter documents.

17. The method of claim 16, further comprising training the LLM by performing training operations comprising:
obtaining a batch of training data comprising a first set of the patterns of the plurality of clinical encounter transcripts;
processing the first set of the patterns of the plurality of clinical encounter transcripts by the LLM to generate an estimated set of post patient encounter documents;
computing a loss based on a deviation between the estimated set of post patient encounter documents and the patterns of post patient encounter documents associated with the first set of the patterns of the plurality of clinical encounter transcripts; and
updating one or more parameters of the LLM based on the computed loss.

18. A system comprising:
one or more processors coupled to a memory comprising non-transitory computer instructions that when executed by the one or more processors perform operations comprising:
receiving a transcript representing a clinical encounter between a patient and a clinician;
analyzing the transcript using a generative machine learning model to automatically generate a preliminary post patient encounter document that comprises patient information and information about the clinical encounter; and
generating, for display to the clinician, a graphical user interface (GUI) comprising the automatically generated preliminary post patient encounter document.

19. The system of claim 18, wherein the preliminary post patient encounter document comprises a subjective objective assessment plan (SOAP) note, a problem, intervention, and evaluation (PIE) note, a data, assessment, and plan (DAP) note, a behavior, intervention, response, and plan (BIRP) note, a follow-up, outcomes, care, upcoming visits, and symptoms (FOCUS) note, or a chief compliant, history, assessment, treatment, and test results (CHART) note, or an intervention, assessment, and plan (IAP) note.

20. A non-transitory computer readable medium comprising non-transitory computer-readable instructions for performing operations comprising:
receiving a transcript representing a clinical encounter between a patient and a clinician;
analyzing the transcript using a generative machine learning model to automatically generate a preliminary post patient encounter document that comprises patient information and information about the clinical encounter; and
generating, for display to the clinician, a graphical user interface (GUI) comprising the automatically generated preliminary post patient encounter document.
